(19) United States
(12) Patent Application Publication
(10) Pub. No.: US 2014/0147532 A1
(43) Pub. Date: May 29, 2014

(54) METHOD OF MANUFACTURING HEALTHY DRINK USING OPUNTIA FICUS-INDICA

(71) Applicant: Seon Ae YUN, Pohang-si (KR)

(72) Inventor: Seon Ae YUN, Pohang-si (KR)

(21) Appl. No.: 13/687,388

(22) Filed: Nov. 28, 2012

(51) Int. Cl. A61K 36/33

(52) U.S. Cl. CPC A61K 36/33
USPC 424/767

(57) ABSTRACT

Provided is a method of manufacturing healthy drink using Opuntia ficus-indica, which includes the steps of: removing fruits and thorns from stems of the Opuntia ficus-indica, washing the stems of the Opuntia ficus-indica, and mixing and grinding 90% to 99.5% by weight of the washed stems of the Opuntia ficus-indica and 0.5% to 10% by weight of roasted ginkgo nuts to thereby prepare a crude liquid of mixed materials; mixing 50% to 60% by weight of the crude liquid of mixed materials and 40% to 50% by weight of water; ripening and filtering the diluted crude liquid for 12 hours to 24 hours at temperature of 60° C. to 80° C.; mixing 0.2% to 1% by weight of at least one of pure crystalline fructose and fructooligosaccharine to 99% to 99.8% by weight of the filtered crude liquid; and sterilizing the crude liquid.

### BACKGROUND OF THE INVENTION

1. Field of the Invention

The present invention relates to a method of manufacturing healthy drink, and more particularly, to a method of manufacturing healthy drink whose components are Opuntia ficus-indica having medical effects of anti-inflammatory action and pain relief, ginkgo nuts, pure crystalline fructose, and fructooligosaccharine, so that consumers can easily take in the Opuntia ficus-indica having excellent medical effects.

2. Background Art

The main component of Opuntia ficus-indica is 88% to 95% of water, and the Opuntia ficus-indica contains dietary fiber, vitamin, and minerals, such as calcium and magnesium, which are good for the human body, pectin for reducing cholesterol or fat content becoming the cause of adult diseases, and phenol and flavonoids to reduce the risk of cancer.

Moreover, nutritional and medical effects of the Opuntia ficus-indica have been generally known through studies of many years. Particularly, it has been well known that the Opuntia ficus-indica provides anti-inflammatory action and pain relief, relieves bronchial asthma, alleviates fever, treats burns, stimulates the circulation of blood, suppresses diabetes and cholesterol, activates the intestines to prevent constipation, has a diuretic effect, and it has been reported that stems of the Opuntia ficus-indica are more effective in such medical effects than fruits of the Opuntia ficus-indica.

However, differently from the fruits having a refreshing taste, the stems of the Opuntia ficus-indica have a sickening smell and a strong bitter taste due to terpene contained therein, and so, they are not popularized despite of such excellent medical effects.

### SUMMARY OF THE INVENTION

Accordingly, the present invention has been made to solve the above-mentioned problems occurring in the prior arts, and it is an object of the present invention to provide a method of manufacturing healthy drink using Opuntia ficus-indica, which can allow consumers to easily take in stems of the Opuntia ficus-indica having excellent medical effects without any repulsion, and which can promote intrinsic medical effects of the Opuntia ficus-indica to provide the anti-inflammatory effect and relieve bronchial asthma.

In order to achieve the above object, the present invention provides a method of manufacturing healthy drink using Opuntia ficus-indica including: a crude liquid extraction process of removing fruits and thorns from stems of the Opuntia ficus-indica, washing the stems of the Opuntia ficus-indica, and mixing and grinding 90% to 99.5% by weight of the washed stems of the Opuntia ficus-indica and 0.5% to 10% by weight of roasted ginkgo nuts to thereby prepare a crude liquid of mixed materials; a dilution process of mixing 50% to 60% by weight of the crude liquid of mixed materials and 40% to 50% by weight of water; a ripening process of ripening the diluted crude liquid for 12 hours to 24 hours at temperature of 60° C. to 80° C.; a filtering process of removing foreign matters from the ripened crude liquid; an additive mixing process of mixing 0.2% to 1% by weight of at least one of pure crystalline fructose and fructooligosaccharine to 99% to 99.8% by weight of the filtered crude liquid; and a sterilization process of sterilizing the crude liquid to which the additives are mixed.

The method of manufacturing healthy drink using the Opuntia ficus-indica according to the present invention can enhance the customer interest while maintaining the excellent medical effects of the stems of the Opuntia ficus-indica as much as possible, and promote the anti-inflammatory effect, relief of bronchial asthma, and improvement of the circulation of blood by mixing ginkgo nuts of a proper amount.

### DETAILED DESCRIPTION OF THE PREFERRED EMBODIMENT

Hereinafter, a method of manufacturing healthy drink using Opuntia ficus-indica according to a preferred embodiment of the present invention will be described in detail in order of a crude liquid extraction process, a dilution process, a ripening process, a filtering process, an additive mixing process, and sterilization process.

### (1) Crude Liquid Extraction Process

The crude liquid extraction process is a process of making a crude liquid of mixed materials through the steps of removing thorns from the stems of the Opuntia ficus-indica from which fruits having relatively higher palatability are removed, cleaning the stems in running water, cleaning and roasting ginkgo nuts after removing testae and tegmina of the ginkgo nuts, and mixing and grinding 90% to 99.5% by weight of the cleaned stems of the Opuntia ficus-indica and 0.5% to 10% by weight of the roasted ginkgo nuts. In this instance, it has been known that the ginkgo nuts contain nutrients such as carbohydrate and protein, vitamin, and beta-carotene, which the stems of the Opuntia ficus-indica do not contain or lack, and the ginkgo nuts have the medical effects of anti-inflammatory action, relief of bronchial asthma, and improvement of the circulation of blood like the stems of the Opuntia ficus-indica. Therefore, such mixing of the stems of the Opuntia ficus-indica and the ginkgo nuts can enhance such medical effects through complementary and supplementary relationship. Furthermore, because the ginkgo nuts contains a great deal of sugar (about 37.4%) having the sweet taste, they can enhance the customer interest by neutralizing the bitter taste of the Opuntia ficus-indica.

### (2) Dilution Process

The dilution process is a process of mixing 50% to 60% by weight of the crude liquid of mixed materials and 40% to 50% by weight of water so as to allow customers to easily take in by diluting the crude liquid of mixed materials of high viscosity. If the ratio of dilution of water is less than 40% by weight, customers feel stickiness when they take in. On the contrary, if the ratio of dilution of water is more than 50% by weight, the customers can easily take in but cannot sufficiently obtain the medical effects and the intrinsic flavors of the Opuntia ficus-indica and the ginkgo nuts. Accordingly, it is preferable that 50% to 60% by weight of the crude liquid of mixed materials and 40% to 50% by weight of water are mixed. Additionally, the mixing ratio of raw materials in the crude liquid extraction process and the dilution ratio in the dilution process, the customers can pay attention to intake of the ginkgo nuts which have side effects of vomit and difficulty in breathing at the time of hyperingestion of the ginkgo nuts.

### (3) Ripening Process

The ripening process is a process of ripening the diluted crude liquid for 12 hours to 24 hours at temperature of 60° C. to 80° C. Through the ripening process, the crude liquid is homogenized. In this instance, if the ripening temperature is lower than 60° C. or the ripening period of time is shorter than 12 hours, the crude liquid is not sufficiently homogenized, but if the ripening temperature is higher than 60° C. or the ripening period of time is longer than 12 hours, nutritional contents of the crude liquid may be destroyed or changed. In the meantime, it is preferable that the crude liquid is stirred continuously during the ripening process.

### (4) Filtering Process

The filtering process is a process of filtering foreign matters, such as peels of the stems of the Opuntia ficus-indica and shells of the ginkgo nuts, contained in the ripened crude liquid. In the filtering process, it is effective to heat the crude liquid at temperature of 60° C. to 80° C. to lower the viscosity.

### (5) Additive Mixing Process

The additive mixing process is a process of enhancing the taste and the customer interest by mixing 0.2% to 1% by weight of at least one of pure crystalline fructose and fructooligosaccharine to 99% to 99.8% by weight of the filtered crude liquid. In this instance, the pure crystalline fructose has a strongly sweet taste and allows diabetics to take in it differently from glucose, and fructooligosaccharine is hardly decomposed by digestive enzymes and ferments by germs in the large intestine to thereby reduce harmful germs and induces useful bifidobacteria to proliferate. Therefore, the method of manufacturing healthy drink using the Opuntia ficus-indica according to the present invention enhances the sense of taste and increases medical effects by additionally mixing pure crystalline fructose or fructooligosaccharine.

### (6) Sterilization Process

The sterilization process is a process of finishing healthy drink by sterilizing the crude liquid to which the above-mentioned additives are mixed. In detail, the crude liquid is heated at temperature of 100° C. to 140° C. for 2 seconds to 15 seconds, and then, is rapidly cooled. In this instance, it is preferable that the crude liquid is re-filtered and packed before the rapid cooling.

### Embodiment

600 g of stems of Opuntia ficus-indica from which fruits are removed was prepared. Thorns of the Opuntia ficus-indica were completely removed, and then, the stems of the Opuntia ficus-indica were washed in water. 50 g of ginkgo nuts from which testae and tegmina were removed were roasted, and then, the stems of the Opuntia ficus-indica and the roasted ginkgo nuts were mixed in a mixer and grinded finely till solid ingredients were all removed, such that the mixed crude liquid was made. Moreover, 650 g of the mixed crude liquid was diluted in 550 g of purified water and ripened at temperature of 70° C. for 15 hours, and then, 1200 g of the ripened crude liquid was filtered through a filter. After that, 5 g of pure crystalline fructose and 5 g of fructooligosaccharine were added to the crude liquid and sterilized at temperature of 100° C. for 5 seconds, and then, was cooled. Through the above processes, healthy drink using the Opuntia ficus-indica was manufactured.

### Compared Example 1

After thorns and peels were removed from the stems of the Opuntia ficus-indica, the stems of the Opuntia ficus-indica were washed in running water, and then, cut into small pieces. After that, the cut stems were put and grinded in the mixer together with a small quantity of mineral water, such that Opuntia ficus-indica juice was made.

### Compared Example 2

Stems of the washed Opuntia ficus-indica were cooled and dried, and then, grinded in a grinder, such that Opuntia ficus-indica powder was made.

### Compared Example 3

100 g of stems of the washed Opuntia ficus-indica were put in 500 ml of water and boiled till the water was reduced in half, such that Opuntia ficus-indica extract was made.

### Sensory Test of Opuntia Ficus-Indica Healthy Drink

In order to evaluate palatability of the Opuntia ficus-indica products manufactured according to the embodiment and the compared examples 1 to 3, 50 persons for the sensory test who were not related with the present invention were selected and the sensor test was carried out in connection with taste, scent, color, and general palatability, which are sensory characteristics, and results of the test was indicated in the following Table 1 . Here, the five-point scale palatability test was used for the sensory test, and in the following Table 1 , sensory grades were indicated in average values, where 1 : very bad, 2: bad, 3: normal, 4: good, 5: very good.

TABLE 1

| | | | | General |
|---|---|---|---|---|
| Div. | Taste | Scent | Color | palatability |
| Embodiment | 4.3 | 4.1 | 3.7 | 4.0 |
| Compared | 1.7 | 2.5 | 4.1 | 2.8 |
| example 1 | | | | |
| Compared | 2.2 | 2.6 | 3.5 | 2.8 |
| example 2 | | | | |
| Compared | 2.3 | 3.0 | 3.2 | 2.8 |
| example 3 | | | | |

As the results of the sensory test, the healthy drink using the Opuntia ficus-indica according to the embodiment of the present invention was superior to the compared examples in the aspect of palatability, such as taste, scent, and so on.

What is claimed is:

1. A method of manufacturing healthy drink using Opuntia ficus-indica comprising:
a crude liquid extraction process of removing fruits and thorns from stems of the Opuntia ficus-indica, washing the stems of the Opuntia ficus-indica, and mixing and grinding 90% to 99.5% by weight of the washed stems of the Opuntia ficus-indica and 0.5% to 10% by weight of roasted ginkgo nuts to thereby prepare a crude liquid of mixed materials;
a dilution process of mixing 50% to 60% by weight of the crude liquid of mixed materials and 40% to 50% by weight of water;
a ripening process of ripening the diluted crude liquid for 12 hours to 24 hours at temperature of 60° C. to 80° C.;
a filtering process of removing foreign matters from the ripened crude liquid;
an additive mixing process of mixing 0.2% to 1% by weight of at least one of pure crystalline fructose and fructooligosaccharine to 99% to 99.8% by weight of the filtered crude liquid; and
a sterilization process of sterilizing the crude liquid to which the additives are mixed.

2. The method of manufacturing healthy drink according to claim 1, wherein in the filtering process, the crude liquid is filtered in a state where it is lowered in viscosity by being heated at temperature of 60° C. to 80° C.

3. The method of manufacturing healthy drink according to claim 1, wherein in the sterilization process, the crude liquid is heated at temperature of 100° C. to 140° C. for 2 seconds to 15 seconds and is rapidly cooled.
